(19) United States
(12) Patent Application Publication
(10) Pub. No.: US 2024/0029863 A1
(43) Pub. Date: Jan. 25, 2024

(54) SYSTEM AND METHOD FOR MANAGING STORAGE AND IMAGE INTERPRETATION

(71) Applicant: Dell Products L.P., Round Rock, TX (US)

(72) Inventors: OFIR EZRIELEV, Be'er Sheva (IL); AMIHAI SAVIR, Newton, MA (US); Oshry Ben-Harush, Cedar Park, TX (US)

(21) Appl. No.: 17/872,918

(22) Filed: Jul. 25, 2022

(51) Int. Cl. G16H 30/20; G06T 7/11; G06F 16/583

(52) U.S. Cl. CPC G16H 30/20; G06T 7/11; G06F 16/583

(57) ABSTRACT

Methods and systems for managing storage of data are provided. To manage storage of data, images may be stored along with data usable to facilitate subsequent interpretation of the images. The data may provide information to a subsequent interpreter regarding how a previous outcome was made using the image. The data may allow the subsequent interpreter to understand how the previous interpreter viewed portions of the image, the order in which the portion of the images were viewed by the previous interpreter, etc. The subsequent interpreter may be provided with context regarding landmarks or other features of the image added by the previous interpreter and identified by the previous interpreter as being relevant to the outcome.

### FIELD

Embodiments disclosed herein relate generally to data storage. More particularly, embodiments disclosed herein relate to systems and methods to manage the storage and use of images.

### BACKGROUND

Computing devices may provide computer-implemented services. The computer-implemented services may be used by users of the computing devices and/or devices operably connected to the computing devices. The computer-implemented services may be performed with hardware components such as processors, memory modules, storage devices, and communication devices. The operation of these components and the components of other devices may impact the performance of the computer-implemented services.

### BRIEF DESCRIPTION OF THE DRAWINGS

Embodiments disclosed herein are illustrated by way of example and not limitation in the figures of the accompanying drawings in which like references indicate similar elements.

FIG. 1 shows a block diagram illustrating a system in accordance with an embodiment.

FIG. 2 shows a diagram illustrating data flow and processes performed by a system in accordance with an embodiment.

FIG. 3A shows a flow diagram illustrating a method of storing and using an image in accordance with an embodiment.

FIG. 3B shows a flow diagram illustrating a method of obtaining data packages and/or videos in accordance with an embodiment.

FIGS. 4A-4H show diagrams illustrating a system, operations performed thereby, and data structures used by the system over time in accordance with an embodiment.

FIG. 5 shows a block diagram illustrating a data processing system in accordance with an embodiment.

### DETAILED DESCRIPTION

Various embodiments will be described with reference to details discussed below, and the accompanying drawings will illustrate the various embodiments. The following description and drawings are illustrative and are not to be construed as limiting. Numerous specific details are described to provide a thorough understanding of various embodiments. However, in certain instances, well-known or conventional details are not described in order to provide a concise discussion of embodiments disclosed herein.

Reference in the specification to “one embodiment” or “an embodiment” means that a particular feature, structure, or characteristic described in conjunction with the embodiment can be included in at least one embodiment. The appearances of the phrases “in one embodiment” and “an embodiment” in various places in the specification do not necessarily all refer to the same embodiment.

In general, embodiments disclosed herein relate to methods and systems for managing storage of data. To manage storage of data, images may be stored along with data usable to facilitate subsequent interpretation of the images.

The data may provide information to a subsequent interpreter regarding how a previous outcome (e.g., medical diagnosis) was made using the image. For example, the data may allow the subsequent user to understand how the previous interpreter viewed portions of the image, the order in which the portion of the images were viewed, etc.

By doing so, the subsequent interpreter may be provided with context regarding landmarks or other features of the image added by the previous interpreter and identified by the previous interpreter as being relevant to the outcome. Consequently, the subsequent interpreter may utilize smaller amounts of the image for the subsequent interpretation (e.g., when compared to attempting to subsequently interpret the image without the additional information). Accordingly, the computing resources expended for the subsequent interpretation may be reduced, the subsequent interpretation may be performed more quickly, and/or other benefits may be obtained.

In an embodiment, a method for managing image interpretation is provided. The method may include identifying initiation of an interpretation of an image by an interpreter; while the interpretation of the image is performed, monitoring: interest indications in regions of the image from the interpreter, and features of the image identified by the interpreter during the interpretation; obtaining an interpretation data package based on the interest indications and the identified features of the image; and during a subsequent interpretation of the image, displaying a moving visual media based on the interpretation data package to direct attention of a subsequent interpreter to a subset of the regions of the image.

The moving visual media may be a movie, video or other media that conveys an order of review of the regions of the image during the interpretation of the image by the interpreter.

The method may also include obtaining a view path based on the interest indications in the regions of the image; and obtaining a focus point along the view path based on the interest indications in the regions of the image; and generating a frame of the moving visual media based on the view path and focus point.

The frame may depict a portion of a diagnostically relevant landmark designated by the interpreter. The moving visual media may depict an order in which the diagnostically relevant landmark and a second diagnostically relevant landmark were designated by the interpreter. The moving visual media may depict an order in which the interpreter viewed the regions preceding designation of the diagnostically relevant landmark.

The view path may indicate an order in which the regions of the image were viewed by the interpreter during the interpretation.

A non-transitory media may include instructions that when executed by a processor cause the computer-implemented method to be performed.

A data processing system may include the non-transitory media and a processor, and may perform the computer-implemented method when the computer instructions are executed by the processor.

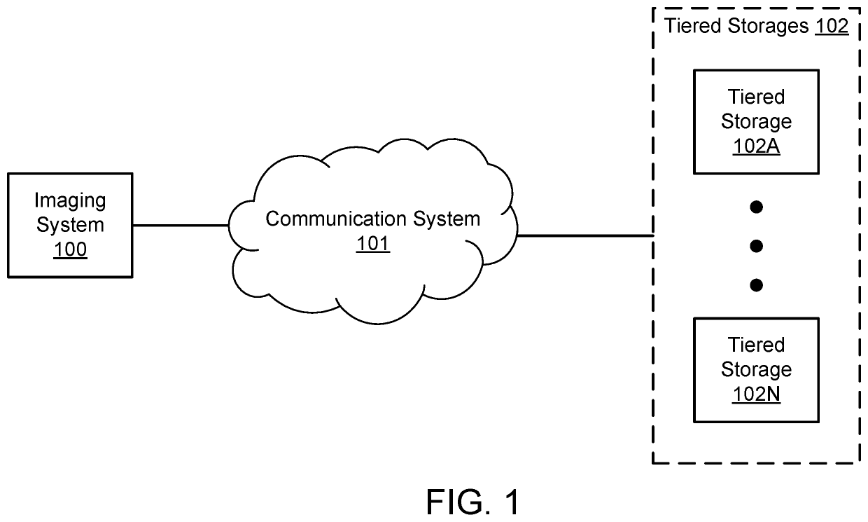

Turning to FIG. 1, a block diagram illustrating a system in accordance with an embodiment is shown. The system shown in FIG. 1 may provide computer-implemented services that may utilize images (e.g., image files) as part of the provided computer-implemented services.

The images may include, for example, super resolution images or other types of images of large size (and/or other sizes). The images may depict various types of scenes which may be useful for a range of purposes. For example, the images may depict scenes useful for medical diagnosis, accident analysis, surveying, and/or other types of purposes.

The computer-implemented services may include any type and quantity of computer-implemented services. The computer-implemented services may include, for example, (i) data storage services for storing and providing copies of the images over time, (ii) analysis services through which the images may be analyzed and information derived from the images may be obtained, and/or (iii) any other type of computer-implemented services that may be performed, at least in part, using images (e.g., image files).

To facilitate use of the images as part of the computer-implemented services, the images may be stored in non-transitory storage for long term retention and/or in memory during use in the computer-implemented services. Due to the size of the images, performance of data processing systems may be less desirable than that desired for the computer-implemented services due to consumption of computing resources for use of the images in the computer-implemented services.

In general, embodiments disclosed herein may provide methods, systems, and/or devices for managing storage and/or use of images to facilitate performance of desired computer-implemented services. To manage storage and use of the images, (i) the images may be segmented and/or stored in tiered storage 102, and (ii) information regarding previous processes performed using the images may be stored to speed subsequent use of the images and/or reduce reading of images from tiered storages 102. By doing so, embodiments disclosed herein may provide a more responsive system by improving the efficiency of resource allocation for accessing images while limiting cost incurred for responsiveness of the system.

To obtain, segment, and/otherwise process the images (e.g., interpret the images), the system of FIG. 1 may include imaging system 100. Imaging system may obtain images, process the images, and/or store the images (and/or portions thereof) in storages (e.g., tiered storages 102). Imaging system 100 may generate the images (e.g., using a capture device) or obtain the images from storage (e.g., read) or other devices (e.g., via communication).

To process the images, imaging system 100 may cooperate with a user to explore and annotate the images. To allow the user to explore the images, imaging system 100 may include a graphical user interface through which a user may select and view portions of images, as well as add annotations such as landmarks (e.g., metadata indicating features of the image) or areas of interest (e.g., metadata indicate a portion of the image).

While the user is exploring and annotating the image, imaging system 100 may track the user's (e.g., an interpreter) interest in various regions (e.g., sub-portions) of the image. For example, imaging system 100 may track (i) regions of the image viewed by the user, (ii) durations of view time for each region, (iii) the user's activities while a region is views such as mouse overs and/or annotations added by the user, (iv) repetitive views of regions, (v) regions not viewed by the user, (vi) patterns in the viewing of the image by the user, and/or (vii) other information which may indicate a user's relative interest level in each of the regions of the image. For example, unviewed regions may indicate the user's lowest relative interest level while long view durations/repetitive viewings/addition of annotations to regions may indicate a user's highest level of interest.

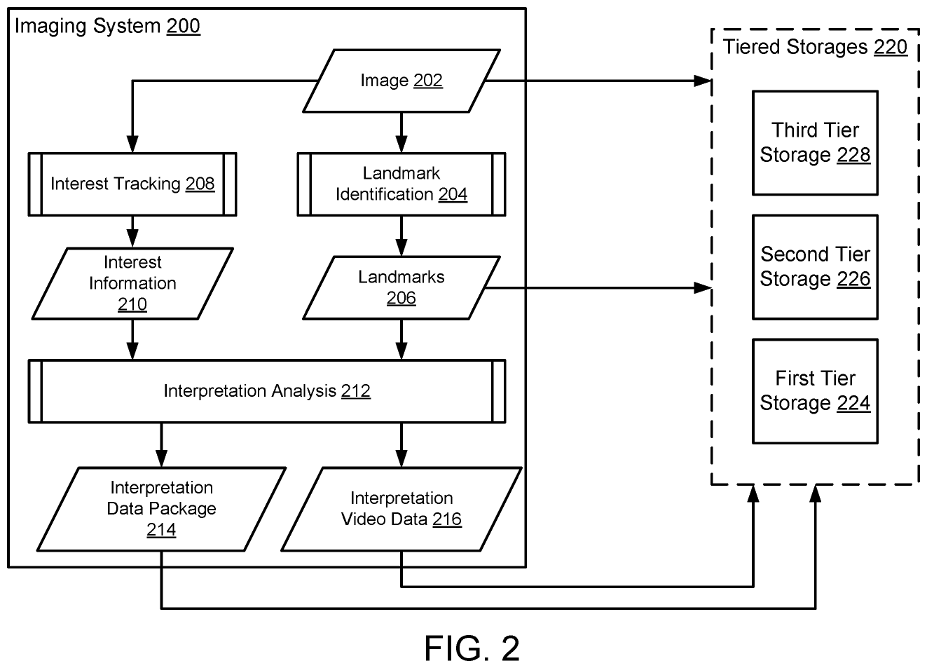

Through monitoring of the user's exploration and annotation of the image, the system may obtain information that may be used to speed subsequent use of the images and/or reduce reading of the stored image in the future. For example, an interpretation data package for an image may be established based on the monitoring of the user's exploration and annotation. The data package may guide subsequent use of the image. By doing so, subsequent users of the image may (i) be provided with information relevant to a subsequent use (e.g., a reinterpretation of the image) of the image and (ii) reduce the likelihood of less relevant portions of the image for subsequent uses from being read. Accordingly, expenditures of computational resources of subsequent uses of the images may be reduced. Refer to FIG. 2A for additional details regarding monitoring use of an image and development of data usable to guide subsequent use of the image in the future.

The aforementioned approach may be usable in a range of different contexts. For example, consider a scenario in which an image is a medical image and the interpreter is a subject matter expert tasked with diagnosing whether a medical condition is indicated by the image. To make the diagnosis, the subject matter expert may review regions of the image to identify patterns or other indicators of the presence, or lack, of the medical condition. Consequently, the subject matter expert may explore the image and annotate the image with landmarks (e.g., identified features, areas of interest, etc.). Once a medical condition is diagnosed, the diagnosis may be reviewed through subsequent interpretation of the image by another subject matter expert or through automated means (e.g., a trained machine learning model trained to diagnose medical conditions using images). During the subsequent interpretation, however, the second subject matter expert may only have the annotations provided by the first interpreter to guide their reinterpretation. Due to the limited number of annotations that the first interpreter may have made, information that may be relevant to the subsequent reinterpretation may be lost. Consequently, the subsequent interpreter may need to exhaustively review the image to perform a clinically appropriate diagnosis.

In contrast, the disclosed embodiments may reduce information loss thereby improving the ability of the subsequent interpreter to reinterpret the image without needing to as exhaustively review the image. For example, when an interpretation is initiated, the subsequent interpreter may be automatically prompted regarding information regarding the previously performed interpretation of the image. If the subsequent interpreter elects to review the previous exploration, a previously generated interpretation data package may be used to provide the subsequent interpreter with information regarding the previous exploration. The interpretation data package may be used to play a video, animation, and/or other depiction of the exploration process performed by the previous interpreter. Through introduction of this additional information, the subsequent interpreter may be able to more efficiently confirm or deny the previous diagnosis made using the image, and/or may be used to facilitate training of other subject matter experts. The second diagnosis may be made by reviewing a smaller number of regions of the image. Consequently, the total quantity of the image necessary to be read from storage for the subsequent interpretation may be reduced.

Tiered storages 102 may store image segments and/or other data structures such as interpretation data packages, moving visual mediums (e.g., videos), and/or other information usable for subsequent use of images. Tiered storages 102 may include any number of tiered storages (e.g., 102A, 102N). Different tiered storages may provide different quality levels with respect to storing data and/or providing copies of stored data. For example, different tiered storages may be implemented with different types and/or quantities of hardware devices. Consequently, different storage tiers may be more or less costly to implement depending on hardware/software components used to implement the storage tiers. To manage cost, tiered storages 102 may include tiered storages with different levels of performance and associated cost. Accordingly, imaging system 100 may store image segments that are more likely to be accessed in the future in higher performance storage tiers (which may have higher associated costs) and other image segments that are less likely to be accessed in the future in lower performance storage tiers.

In an embodiment, tiered storages 102 is implemented with a range of different storage tiers providing different levels of performance having corresponding levels of associated cost. Thus, the image segments may be distributed to the different storage tiers based on corresponding likelihoods of future access. The likelihood of future access may depend on whether use of the image segment is implicated by an interpretation data package, interpretation video data, and/or other factors.

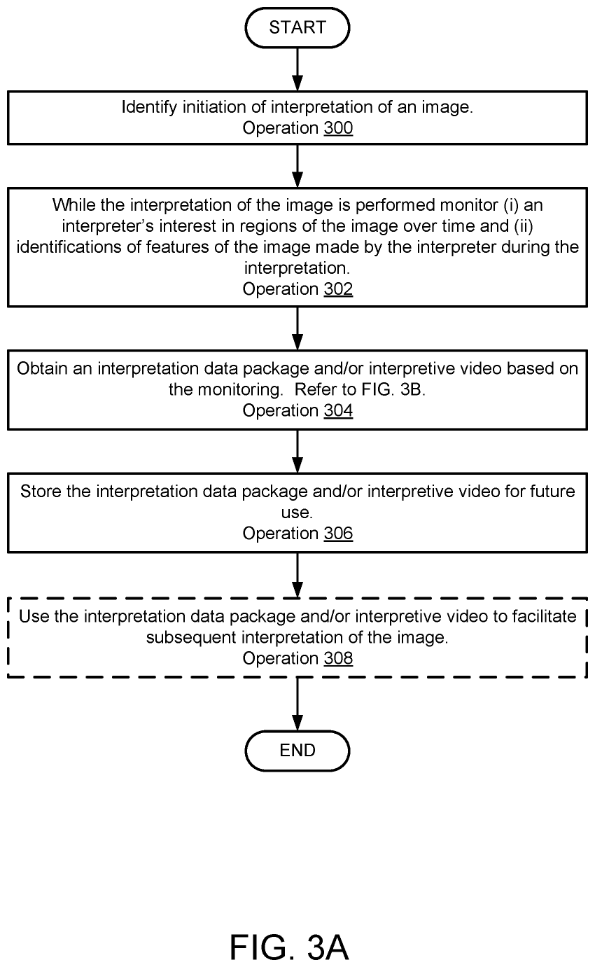

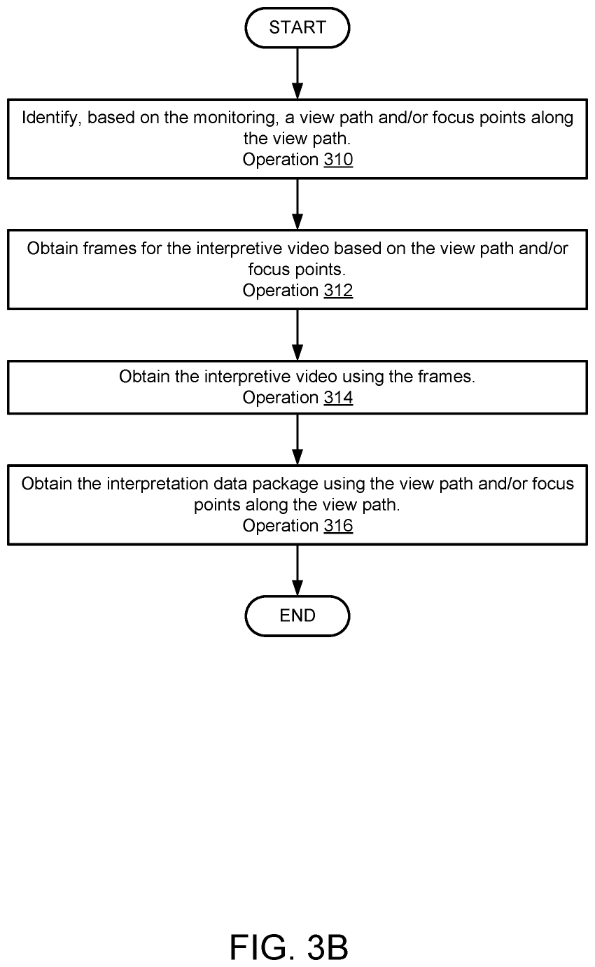

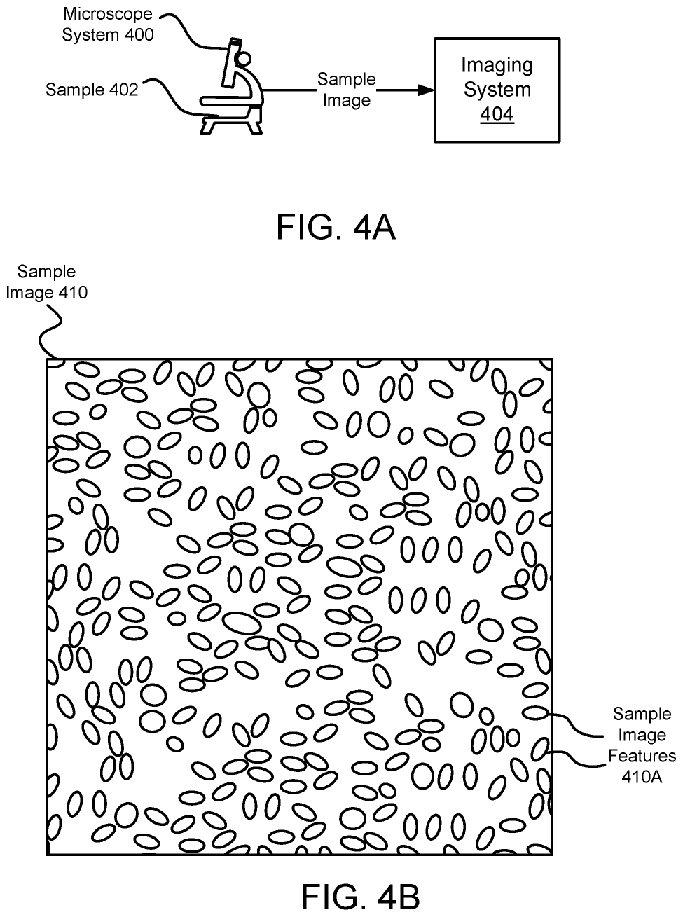

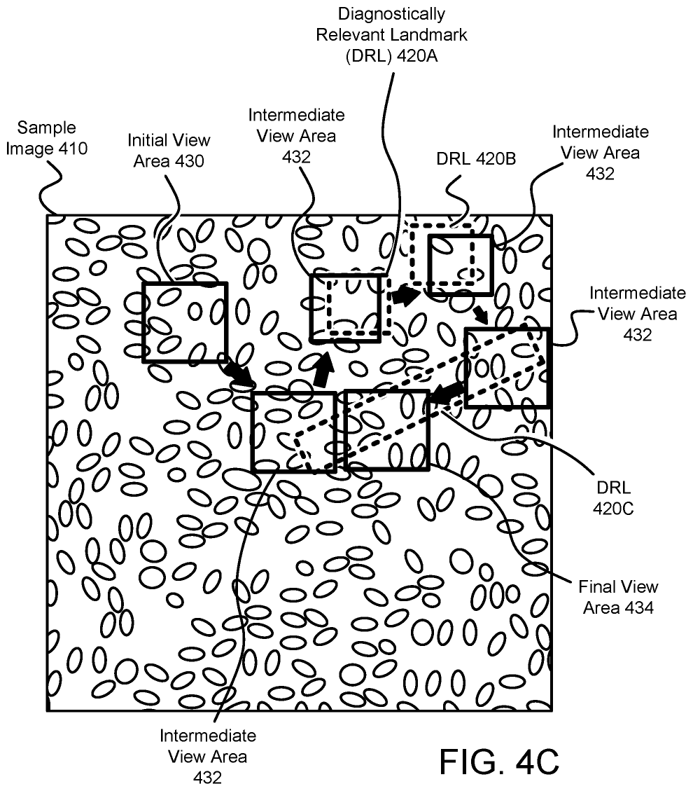

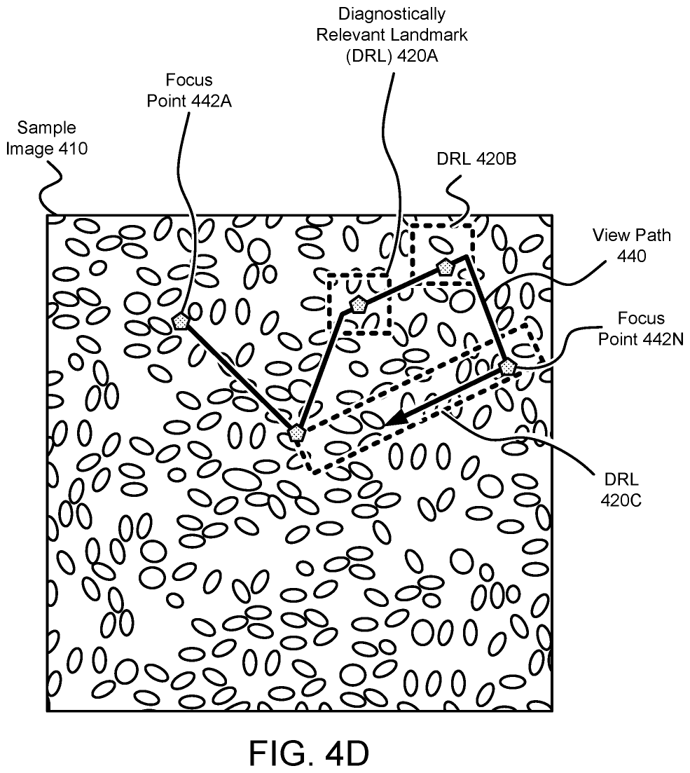

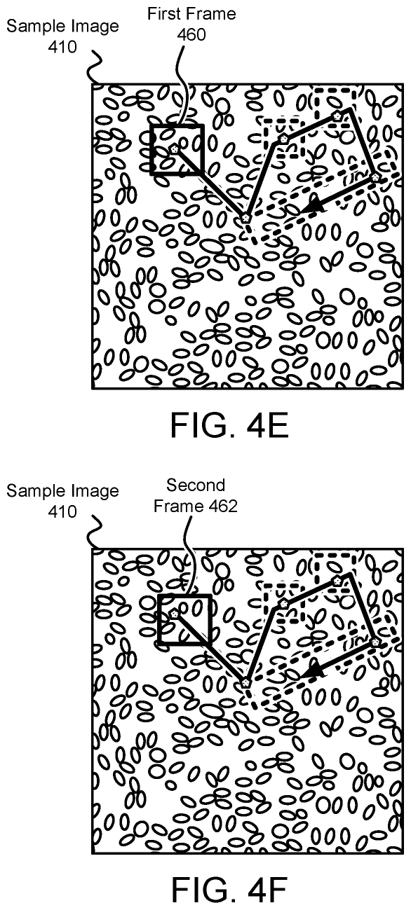

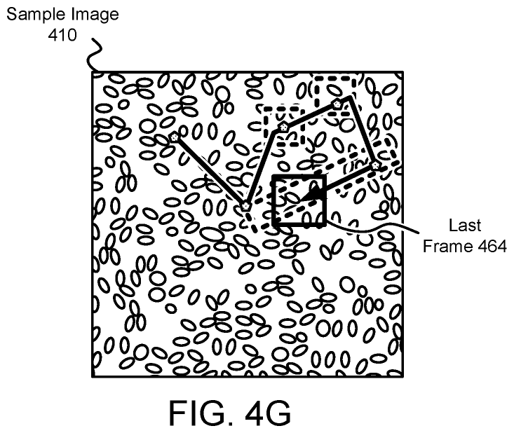

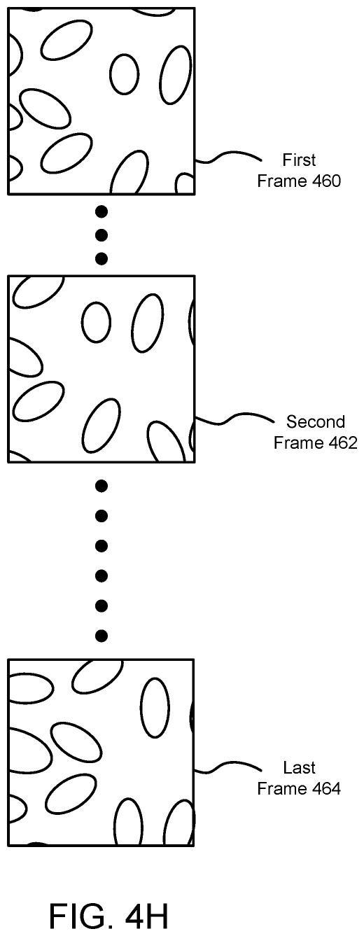

When performing its functionality, one or more of imaging system 100 and tiered storages 102 may perform all, or a portion, of the methods and/or actions shown in FIGS. 3A-4H.

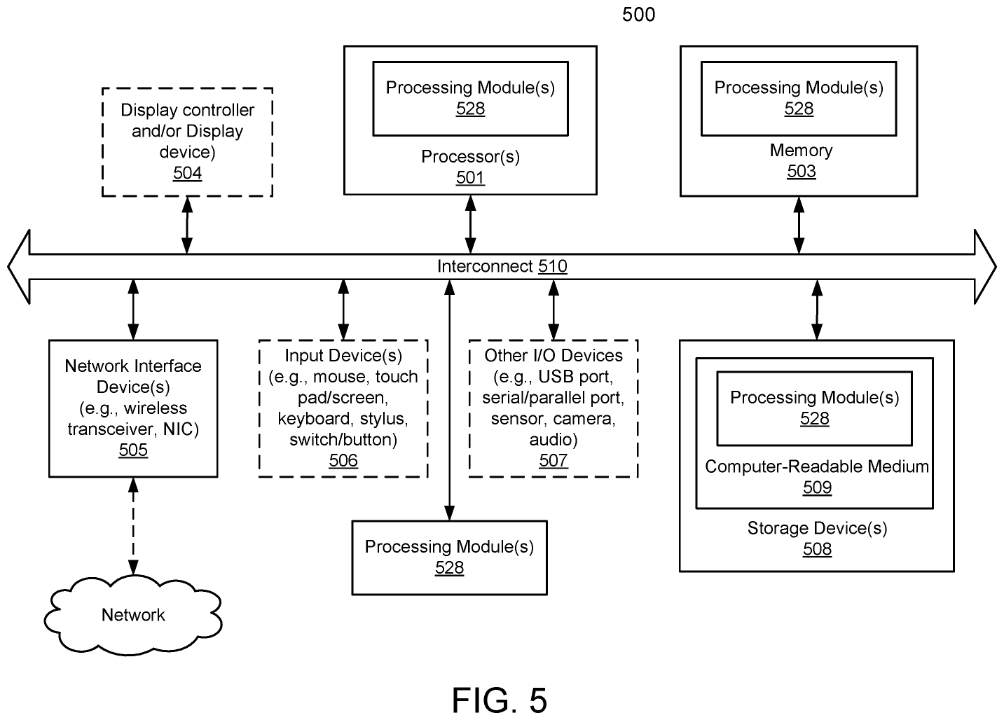

Any of imaging system 100 and tiered storages 102 may be implemented using a computing device (e.g., a data processing system) such as a host or a server, a personal computer (e.g., desktops, laptops, and tablets), a “thin” client, a personal digital assistant (PDA), a Web enabled appliance, a mobile phone (e.g., Smartphone), an embedded system, local controllers, an edge node, and/or any other type of data processing device or system. For additional details regarding computing devices, refer to FIG. 5.

Any of the components illustrated in FIG. 1 may be operably connected to each other (and/or components not illustrated) with a communication system 101.

In an embodiment, communication system 101 includes one or more networks that facilitate communication between any number of components. The networks may include wired networks and/or wireless networks (e.g., and/or the Internet). The networks may operate in accordance with any number and types of communication protocols (e.g., such as the internet protocol).

In an embodiment, communication system 101 is implemented with one or more local communications links (e.g., a bus interconnecting a processor of imaging system 100 and any of the tiered storages).

While illustrated in FIG. 1 as included a limited number of specific components, a system in accordance with an embodiment may include fewer, additional, and/or different components than those illustrated therein.

Turning to FIG. 2, a data flow diagram in a system similar to that illustrated in FIG. 1 in accordance with an embodiment is shown. Imaging system 200 may be similar to imaging system 100, and tiered storages 220 may be similar to tiered storage 102.

Imaging system 200 may obtain image 202. Image 202 may be a data structure including information regarding a scene. For example, image 202 may be any type of image file. The image file may include lossy or lossless compression, may be of any family type (e.g., raster, vector, etc.) or a hybrid, and may include any quantity of information regarding a scene. The image file may be of any format (e.g., Joint Photographic Experts Group (JPEG), Tagged Image File Format (TIFF), Portable Network Graphics (PNG), Graphics Interchange Format (GIF), etc.). Image 202 may be obtained by receiving it from another device (e.g., an imaging device such as a camera), reading it from storage, or by generating it using an imaging device.

Imaging system 200 may perform landmark identification 204 and/or interest track 208 for image 202. These operations may generate data structures used to select storage location(s) for image 202, and/or store image 202.

Landmark identification 204 may identify one or more landmarks 206 in image 202. Landmarks 206 may correspond to features or regions (e.g., groups of pixels corresponding to portions of the depicted scene) of image 202. Landmarks 206 may be implemented with metadata which may indicate the pixels, features, areas of interest, and corresponding portions of image 202.

In an embodiment, landmark identification 204 is performed in cooperation with a subject matter expert. Imaging system 200 may display a graphical user interface and exploration controls (e.g., pan, zoom, etc.) that may allow the user to explore the image. The graphical user interface may also include annotation tools that allow the user to identify landmarks 206 in the image. For example, the annotation tools may allow the user to select portions of the image, designate information regarding the selected portions (e.g., landmark name, type, etc.), and/or perform other functions through which landmarks 206 may be obtained. The user may input user input through these tools and imaging system 200 may use the input to generate metadata corresponding to landmarks 206. The landmarks 206 may correspond to a purpose for which the user is exploring the image, such as to establish a medical diagnosis. The landmarks may be viewed, for example, as notes from the user supporting the medical diagnoses. However, landmarks 206 may not capture all of the information used by the user to make the medical diagnosis. For example, the user may view various parts of image 202 in certain orders, repetitively, etc. which may not be conveyed to a subsequent interpreter through landmarks 206.

While landmark identification 204 is performed, interest tracking 208 process may monitor landmark identification 204 to identify regions of image 202 viewed by the interpreter, orders of viewing the regions, time spend viewing the regions, and/or other information regarding landmark identification 204 which may not be reflected in landmarks 206. For example, interest tracking 208 may monitor information usable to generate a video that depicts the process through which the user reviewed image 202 to come up with a medical diagnosis or make other types of decisions. Consequently, a subsequent viewer of image 202 may review the video to obtain additional information regarding the previous review of image 202 by the first user.

Interest tracking 208 may generate interest information 210, which may include identifiers of regions of image 202, review durations of the regions of images 202, and/or any other types of information collected during interest tracking 208.

Landmarks 206 may be stored in tiered storages 220 along with image 202. Landmarks 206 and interest analysis 212 may be further processed during interpretation analysis 212. Interpretation analysis 212 may be performed to obtain various data structures usable during subsequent interpretation of image 202. The data structures may include interpretation data package 214 and interpretation video data 216.

Interpretation data package 214 may include information usable to synthesize a video or other type of moving visual media to convey information regarding a previous review of image 202. For example, interpretation data package 214 may include instructions usable to generate video frames using image segments stored in tiered storages 220.

Interpretation video data 216 may include a video or other type of moving visual media to convey information regarding a previous review of image 202.

Either of these data structures may also be stored in tiered storages 220.

When subsequent use of image 202 is initiated, interpretation data package 214 and/or interpretation video data 216 may be used to speed the subsequent use and/or reduce the amount of data read from tiered storages 220, as discussed above.

As discussed above, the components of FIG. 1 may perform various methods to manage use of images. FIGS. 3A-3B illustrate methods that may be performed by the components of FIG. 1. In the diagram discussed below and shown in FIGS. 3A-3B, any of the operations may be repeated, performed in different orders, and/or performed in parallel with or in a partially overlapping in time manner with other operations.

Turning to FIG. 3A, a flow diagram illustrating a method of interpreting an image in accordance with an embodiment is shown. The method may be performed by an imaging system or another data processing system.

At operation 300, initiation of interpretation of an image is identified. The identification may be made when a graphical use interface through which the image is viewed is launched. Prior to operation 300, the image may be obtained, for example, by reading it from storage or generating it with an image capture device (e.g., a camera).

At operation 302, while the interpretation of the image is being performed, monitoring of (i) an interpreter's interest in regions of the image over time and/or (ii) identifications of features of the image made by the interpreter during the interpretation may be performed. The interpreter's interest in the regions of the image may be monitored using the graphical user interface through which the user views the image. The regions displayed, time of display, and/or other information regarding viewing of the regions may be used to gauge the interpreter's interest in each of the regions.

Like the interpreter's interest level, the identification of features of the image may also be monitored through the graphical user interface. For example, the interpreter may use tools of the graphical user interface to mark the features. The features may be landmarks such as portions of the scene depicted by the image, areas of interest, and/or other portions of the image or information derived from the image. The user input provided by the graphical user interface and corresponding invocations of the annotation functionality of the user interface may be used to monitor the identification of the features by the interpreter.

At operation 304, an interpretation data package and/or interpretive video is obtained based on the monitoring. The interpretation data package and/or interpret video may be obtained via the method illustrated in FIG. 3B.

At operation 306, the interpretation data package and/or interpretative video is stored for future use. The interpretation data package and/or interpretative video may be stored by sending one or more of them to tiered storage for storage, or to another storage for storage.

The method may end following operation 306.

Following operation 306, a subsequent interpretation (or initiation of subsequent interpretation) of the image may be identified as part of operation 308. This identification may trigger prompting of a subsequent interpreter to use the interpretation data package and/or interpretive video to speed subsequent interpretation of the image. For example, one or more of these data structures may be used to present a moving image depiction of the previous interpretation process. By doing so, a subsequent interpreter may be efficiently appraised of both the landmarks identified by the previous interpreter and other information that the previous interpreter used to identify the landmarks/other purposes.

Turning to FIG. 3B, a flow diagram illustrating a method of obtaining an interpretation data package in accordance with an embodiment is shown. The method may be performed by an imaging system or another data processing system.

At operation 310, a view path and/or focus points along the view path are identified based on the monitoring described with respect to FIG. 3A. The view path may be identified by, for example, identifying a central point of each portion of the image viewed by the interpreter over time, and connecting the central point of each portion. In an embodiment, the central points of the portions may be subjected to clustering or other algorithms to identifying groupings of the central points. The central point of the groups of the central points may be connected with, for example, straight lines, splines, or other segments to establish the view path.

The focus points may be identified based on the durations each portion of the image are viewed by the interpreter. For example, the focus points may be established based on a duration threshold which may be static or dynamically set so that a predetermined number of focus points are established. For each portion that meets the threshold, the nearest point to the center of the portion of the image meeting may be designated as a focus point. The focus points may be weighted based on the corresponding viewing durations. Consequently, during viewing of a video based on the view path and focus points, the frames of the video may correspond to views of portions of the image along the view path, with the number of frames for corresponding to the focus points (e.g., to proportionally set durations of review) being based on the weights of the viewpoints. The resulting video may, for example, walk the subsequent interpreter along the view path of the previous interpreter, and include pauses in movement at each of the focus points along the view path.

At operation 312, frames for an interpretive video based on the view path and/or focus points are obtained. The frames may be obtained using the pixels of the image corresponding to portions of the image along the view path. For example, each frame may include a portion of the pixels of the image.

Different frames may include different numbers of images. For example, frames that are not associated with focus points may include fewer numbers of pixels (e.g., reduced resolution) while frames associated with focus points may include larger numbers of pixels (e.g., native resolution).

At operation 314, the interpretive video is obtained using the frames. The interpretive video may be obtained by compiling the frames.

At operation 316, the interpretation data package is obtained using the view path and/or focus points along the view path. The interpretation data package may be obtained by adding information regarding the view path and/or focus points along the view path. The information may allow for an interpretive video to be dynamically generated and/or displayed to a user using an image (or portion thereof).

The method may end following operation 316.

Using the methods illustrated in FIGS. 3A-3B, embodiments disclosed herein may provide a system that stores images in a manner that facilitate subsequent interpretation of the images. In this manner, the quantity of computing resources used during subsequent interpretation may be reduced by providing the subsequent interpreter with additional information regarding previous interpretation of the image.

Turning to FIGS. 4A-4H, diagrams illustrating a process of storing an image and facilitating subsequent interpretation of an image in accordance with an embodiment are shown.

Turning to FIG. 4A, consider a scenario in which a medical image of sample 402 useful for medical diagnosis purposes is obtained using microscope system 400, which may include a camera and some number of lenses uses to project a depiction of sample 402 on a capture device of the camera. The sample image may be obtained by imaging system 404, which may be similar to the imaging system illustrated in FIG. 1.

Turning to FIG. 4B, a diagram of sample image 410 in accordance with an embodiment is shown. Sample image 410 may be complex and include many features regarding a scene. For example, sample 402 may be a tissue sample from a person. In FIG. 4B, the circles within the border of sample image 410 may represent portions of the image corresponding to cells, proteins, and/or other portions of the tissue. To perform a medical diagnosis, the content and structure of these cells, proteins, and/or other portions of the tissue may be analyzed by a subject matter expert. As part of that analysis, the subject matter expert may identify landmarks within the image contributing to a final medical diagnosis. For example, certain formations of cells such as sample features 410A may indicate the presence of cancer or other illnesses.

Turning to FIG. 4C, a second diagram of sample image 410 reflecting an interpretation process in accordance with an embodiment is shown. To interpret sample image 410, the subject matter expert may utilize a graphical user interface to view various portions of the image. The subject matter expert may then use the graphical user interface to pan, rotate, and/or perform other actions to view other portions of the image.

For example, consider a scenario where the subject matter expert uses the graphical user interface to depict initial view area 430 (the outline of the box indicating the extent of the view presented to the subject matter expert). Over time, the subject matter expert may then pan to view any number of intermediate view areas (e.g., 432) until viewing final view area 434. In FIG. 4C, only some of the views seen by the subject matter are depicted using the boxes. The oversized, solid black arrows indicate the general path that the views of sample image 410 followed as the subject matter expert panned through the image.

During this processes, the subject matter expert may identify landmarks including, for example, three diagnostically relevant landmarks 420A, 420B, 420C. For example, the subject matter expert's knowledge may allow the user to identify patterns or features in sample image 410 that are relevant to a medical diagnosis which the subject matter expert is tasked with making. To document and/or facilitate the diagnosis, the subject matter expert may add annotations corresponding to these landmarks so that a basis for the diagnosis may be recorded. However, as seen in FIG. 4C, the subject matter expert only recorded a small amount of information upon which the diagnosis may be based through annotation of the landmarks.

As seen in FIG. 4C, several views of sample image 410 were viewed by the subject matter expert but are not in any way indicated in by sample image 410 and diagnostically relevant landmarks 420A, 420B, 420C. Thus, a subsequent interpreter may be only provided with an indication of a portion of the information that the subject matter expert took into account to make the medical diagnosis.

As discussed above, to provide subsequent interpreters with additional information regarding previous interpretations, the process performed by the subject matter expert may be monitored and used to generate data structures usable to empower subsequent interpretation with more information regarding the previous interpretation.

Turning to FIG. 4D, to do so, view path 440 may be identified, and focus points 422A-422N along view path 440 may be identified. View path 440 may be identified by identifying the center of each of the view areas, and connecting the centers with a line, fitting a line to the centers, through clustering to identify points that may be connected by a line, or via other methods. Focus points 442A-442N may be identified by calculating durations of time the subject matter expert spends observing portions of sample image 410 proximate to the line, based on landmarks (e.g., 420A-420C) proximate to the line, and/or other information.

Once identified, a data package may be established by adding information regarding view path 440 and/or focus points 442A-442N to the data package. The data package may be used to dynamically construct a video. Additionally or alternatively, the video may be generated based on the view path 440 and/or focus points 442A-442N.

To do so, turning to FIG. 4E, first frame 460 may be obtained. First frame 460 may be a view of sample image 410 proximate to a start of view path 440. In FIG. 4E, first frame 460 may include the pixel of sample image 410 indicated by the corresponding boxed portion of sample image 410.

Any number of intermediate frames may then be obtained. For example, turning to FIG. 4F, view path 440 may be walked a predetermined distance, and second frame 462 may be obtained based on the location along view path 440. In FIG. 4F, second frame 462 may include the pixel of sample image 410 indicated by the corresponding boxed portion of sample image 410.

This process may then be repeated until the end of view path 440 is reached. Turning to FIG. 4G, last frame 464 may include the pixel of sample image 410 indicated by the corresponding boxed portion of sample image 410, entered about the end of view path 440.

While walking view path, multiple duplicate frames for the video may be obtained to establish a pause in the video. For example, while walking view path 440, any of the focus points may be reached. When a focus point is reached, multiple frames (e.g., corresponding to a weight for the focus point) may be generated using a same portion of sample image 410 or a pause may be added to continue to view the same frame for a duration of time may be added so that a resulting video pauses for portions of time corresponding to the weights. The frames may generally be stored in higher performance storage, and in an efficient manner (e.g., without duplication).

Turning to FIG. 4H, the frames 460-464 may be aggregated to obtain the video. In this manner, a subsequent interpreter may be provided with additional information regarding a previous interpretation of a sample image. A similar process for obtaining frames may be performed when a data package is used to dynamically generate a video.

While the frames shown in FIG. 4H do not illustrated landmarks, it will be appreciated that any of the frames may include representations of landmarks identified by the subject matter expert. The representations may include, for example, coloring, outlining, pointers, and/or other graphical entities which may direct attention to a portion of an image.

By doing so, embodiments disclosed herein may provide a system that efficiently marshals limited available computing resources for storage and subsequent use of images while limiting cost for storing the images.

Any of the components illustrated in FIGS. 1-4H may be implemented with one or more computing devices. Turning to FIG. 5, a block diagram illustrating an example of a data processing system (e.g., a computing device) in accordance with an embodiment is shown. For example, system 500 may represent any of data processing systems described above performing any of the processes or methods described above. System 500 can include many different components. These components can be implemented as integrated circuits (ICs), portions thereof, discrete electronic devices, or other modules adapted to a circuit board such as a motherboard or add-in card of the computer system, or as components otherwise incorporated within a chassis of the computer system. Note also that system 500 is intended to show a high level view of many components of the computer system. However, it is to be understood that additional components may be present in certain implementations and furthermore, different arrangement of the components shown may occur in other implementations. System 500 may represent a desktop, a laptop, a tablet, a server, a mobile phone, a media player, a personal digital assistant (PDA), a personal communicator, a gaming device, a network router or hub, a wireless access point (AP) or repeater, a set-top box, or a combination thereof. Further, while only a single machine or system is illustrated, the term “machine” or “system” shall also be taken to include any collection of machines or systems that individually or jointly execute a set (or multiple sets) of instructions to perform any one or more of the methodologies discussed herein.

In one embodiment, system 500 includes processor 501, memory 503, and devices 505-507 via a bus or an interconnect 510. Processor 501 may represent a single processor or multiple processors with a single processor core or multiple processor cores included therein. Processor 501 may represent one or more general-purpose processors such as a microprocessor, a central processing unit (CPU), or the like. More particularly, processor 501 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. Processor 501 may also be one or more special-purpose processors such as an application specific integrated circuit (ASIC), a cellular or baseband processor, a field programmable gate array (FPGA), a digital signal processor (DSP), a network processor, a graphics processor, a network processor, a communications processor, a cryptographic processor, a co-processor, an embedded processor, or any other type of logic capable of processing instructions.

Processor 501, which may be a low power multi-core processor socket such as an ultra-low voltage processor, may act as a main processing unit and central hub for communication with the various components of the system. Such processor can be implemented as a system on chip (SoC). Processor 501 is configured to execute instructions for performing the operations discussed herein. System 500 may further include a graphics interface that communicates with optional graphics subsystem 504, which may include a display controller, a graphics processor, and/or a display device.

Processor 501 may communicate with memory 503, which in one embodiment can be implemented via multiple memory devices to provide for a given amount of system memory. Memory 503 may include one or more volatile storage (or memory) devices such as random access memory (RAM), dynamic RAM (DRAM), synchronous DRAM (SDRAM), static RAM (SRAM), or other types of storage devices. Memory 503 may store information including sequences of instructions that are executed by processor 501, or any other device. For example, executable code and/or data of a variety of operating systems, device drivers, firmware (e.g., input output basic system or BIOS), and/or applications can be loaded in memory 503 and executed by processor 501. An operating system can be any kind of operating systems, such as, for example, Windows® operating system from Microsoft®, Mac OS®/iOS® from Apple, Android® from Google®, Linux®, Unix®, or other real-time or embedded operating systems such as VxWorks.

System 500 may further include IO devices such as devices (e.g., 505, 506, 507, 508) including network interface device(s) 505, optional input device(s) 506, and other optional IO device(s) 507. Network interface device(s) 505 may include a wireless transceiver and/or a network interface card (NIC). The wireless transceiver may be a WiFi transceiver, an infrared transceiver, a Bluetooth transceiver, a WiMax transceiver, a wireless cellular telephony transceiver, a satellite transceiver (e.g., a global positioning system (GPS) transceiver), or other radio frequency (RF) transceivers, or a combination thereof. The NIC may be an Ethernet card.

Input device(s) 506 may include a mouse, a touch pad, a touch sensitive screen (which may be integrated with a display device of optional graphics subsystem 504), a pointer device such as a stylus, and/or a keyboard (e.g., physical keyboard or a virtual keyboard displayed as part of a touch sensitive screen). For example, input device(s) 506 may include a touch screen controller coupled to a touch screen. The touch screen and touch screen controller can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with the touch screen.

IO devices 507 may include an audio device. An audio device may include a speaker and/or a microphone to facilitate voice-enabled functions, such as voice recognition, voice replication, digital recording, and/or telephony functions. Other IO devices 507 may further include universal serial bus (USB) port(s), parallel port(s), serial port(s), a printer, a network interface, a bus bridge (e.g., a PCI-PCI bridge), sensor(s) (e.g., a motion sensor such as an accelerometer, gyroscope, a magnetometer, a light sensor, compass, a proximity sensor, etc.), or a combination thereof. IO device(s) 507 may further include an imaging processing subsystem (e.g., a camera), which may include an optical sensor, such as a charged coupled device (CCD) or a complementary metal-oxide semiconductor (CMOS) optical sensor, utilized to facilitate camera functions, such as recording photographs and video clips. Certain sensors may be coupled to interconnect 510 via a sensor hub (not shown), while other devices such as a keyboard or thermal sensor may be controlled by an embedded controller (not shown), dependent upon the specific configuration or design of system 500.

To provide for persistent storage of information such as data, applications, one or more operating systems and so forth, a mass storage (not shown) may also couple to processor 501. In various embodiments, to enable a thinner and lighter system design as well as to improve system responsiveness, this mass storage may be implemented via a solid state device (SSD). However, in other embodiments, the mass storage may primarily be implemented using a hard disk drive (HDD) with a smaller amount of SSD storage to act as a SSD cache to enable non-volatile storage of context state and other such information during power down events so that a fast power up can occur on re-initiation of system activities. Also a flash device may be coupled to processor 501, e.g., via a serial peripheral interface (SPI). This flash device may provide for non-volatile storage of system software, including a basic input/output software (BIOS) as well as other firmware of the system.

Storage device 508 may include computer-readable storage medium 509 (also known as a machine-readable storage medium or a computer-readable medium) on which is stored one or more sets of instructions or software (e.g., processing module, unit, and/or processing module/unit/logic 528) embodying any one or more of the methodologies or functions described herein. Processing module/unit/logic 528 may represent any of the components described above. Processing module/unit/logic 528 may also reside, completely or at least partially, within memory 503 and/or within processor 501 during execution thereof by system 500, memory 503 and processor 501 also constituting machine-accessible storage media. Processing module/unit/logic 528 may further be transmitted or received over a network via network interface device(s) 505.

Computer-readable storage medium 509 may also be used to store some software functionalities described above persistently. While computer-readable storage medium 509 is shown in an exemplary embodiment to be a single medium, the term “computer-readable storage medium” should be taken to include a single medium or multiple media (e.g., a centralized or distributed database, and/or associated caches and servers) that store the one or more sets of instructions. The terms “computer-readable storage medium” shall also be taken to include any medium that is capable of storing or encoding a set of instructions for execution by the machine and that cause the machine to perform any one or more of the methodologies of embodiments disclosed herein. The term “computer-readable storage medium” shall accordingly be taken to include, but not be limited to, solid-state memories, and optical and magnetic media, or any other non-transitory machine-readable medium.

Processing module/unit/logic 528, components and other features described herein can be implemented as discrete hardware components or integrated in the functionality of hardware components such as ASICS, FPGAs, DSPs or similar devices. In addition, processing module/unit/logic 528 can be implemented as firmware or functional circuitry within hardware devices. Further, processing module/unit/logic 528 can be implemented in any combination hardware devices and software components.

Note that while system 500 is illustrated with various components of a data processing system, it is not intended to represent any particular architecture or manner of interconnecting the components; as such details are not germane to embodiments disclosed herein. It will also be appreciated that network computers, handheld computers, mobile phones, servers, and/or other data processing systems which have fewer components or perhaps more components may also be used with embodiments disclosed herein.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Embodiments disclosed herein also relate to an apparatus for performing the operations herein. Such a computer program is stored in a non-transitory computer readable medium. A non-transitory machine-readable medium includes any mechanism for storing information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices).

The processes or methods depicted in the preceding figures may be performed by processing logic that comprises hardware (e.g. circuitry, dedicated logic, etc.), software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

Embodiments disclosed herein are not described with reference to any particular programming language. It will be appreciated that a variety of programming languages may be used to implement the teachings of embodiments disclosed herein.

In the foregoing specification, embodiments have been described with reference to specific exemplary embodiments thereof. It will be evident that various modifications may be made thereto without departing from the broader spirit and scope of the embodiments disclosed herein as set forth in the following claims. The specification and drawings are, accordingly, to be regarded in an illustrative sense rather than a restrictive sense.

What is claimed is:

1. A method for managing image interpretation, the method comprising:
identifying initiation of an interpretation of an image by an interpreter;
while the interpretation of the image is performed, monitoring:
interest indications in regions of the image from the interpreter, and
features of the image identified by the interpreter during the interpretation;
obtaining an interpretation data package based on the interest indications and the identified features of the image; and
during a subsequent interpretation of the image, displaying a moving visual media based on the interpretation data package to direct attention of a subsequent interpreter to a subset of the regions of the image.

2. The method of claim 1, wherein the moving visual media is a movie that conveys an order of review of the regions of the image during the interpretation of the image by the interpreter.

3. The method of claim 2, further comprising:
obtaining a view path based on the interest indications in the regions of the image; and
obtaining a focus point along the view path based on the interest indications in the regions of the image; and
generating a frame of the moving visual media based on the view path and focus point.

4. The method of claim 3, wherein the frame depicts a portion of a diagnostically relevant landmark designated by the interpreter.

5. The method of claim 4, wherein the moving visual media depicts an order in which the diagnostically relevant landmark and a second diagnostically relevant landmark were designated by the interpreter.

6. The method of claim 4, wherein the moving visual media depicts an order in which the interpreter viewed the regions preceding designation of the diagnostically relevant landmark.

7. The method of claim 3, wherein the view path indicates an order in which the regions of the image were viewed by the interpreter during the interpretation.

8. A non-transitory machine-readable medium having instructions stored therein, which when executed by a processor, cause the processor to perform operations for managing image interpretation, the operations comprising:
identifying initiation of an interpretation of an image by an interpreter;
while the interpretation of the image is performed, monitoring:
interest indications in regions of the image from the interpreter, and
features of the image identified by the interpreter during the interpretation;
obtaining an interpretation data package based on the interest indications and the identified features of the image; and
during a subsequent interpretation of the image, displaying a moving visual media based on the interpretation data package to direct attention of a subsequent interpreter to a subset of the regions of the image.

9. The non-transitory machine-readable medium of claim 8, wherein the moving visual media is a movie that conveys an order of review of the regions of the image during the interpretation of the image by the interpreter.

10. The non-transitory machine-readable medium of claim 9, wherein the operations further comprise:
obtaining a view path based on the interest indications in the regions of the image; and
obtaining a focus point along the view path based on the interest indications in the regions of the image; and
generating a frame of the moving visual media based on the view path and focus point.

11. The non-transitory machine-readable medium of claim 10, wherein the frame depicts a portion of a diagnostically relevant landmark designated by the interpreter.

12. The non-transitory machine-readable medium of claim 11, wherein the moving visual media depicts an order in which the diagnostically relevant landmark and a second diagnostically relevant landmark were designated by the interpreter.

13. The non-transitory machine-readable medium of claim 11, wherein the moving visual media depicts an order in which the interpreter viewed the regions preceding designation of the diagnostically relevant landmark.

14. The non-transitory machine-readable medium of claim 10, wherein the view path indicates an order in which the regions of the image were viewed by the interpreter during the interpretation.

15. A data processing system, comprising:
a processor; and
a memory coupled to the processor to store instructions, which when executed by the processor, cause the processor to perform operations for managing image interpretation, the operations comprising:
identifying initiation of an interpretation of an image by an interpreter;
while the interpretation of the image is performed, monitoring:
interest indications in regions of the image from the interpreter, and
features of the image identified by the interpreter during the interpretation;
obtaining an interpretation data package based on the interest indications and the identified features of the image; and
during a subsequent interpretation of the image, displaying a moving visual media based on the interpretation data package to direct attention of a subsequent interpreter to a subset of the regions of the image.

16. The data processing system of claim 15, wherein the moving visual media is a movie that conveys an order of review of the regions of the image during the interpretation of the image by the interpreter.

17. The data processing system of claim 16, further comprising:
obtaining a view path based on the interest indications in the regions of the image; and
obtaining a focus point along the view path based on the interest indications in the regions of the image; and
generating a frame of the moving visual media based on the view path and focus point.

18. The data processing system of claim 17, wherein the frame depicts a portion of a diagnostically relevant landmark designated by the interpreter.

19. The data processing system of claim 18, wherein the moving visual media depicts an order in which the diagnostically relevant landmark and a second diagnostically relevant landmark were designated by the interpreter.

20. The data processing system of claim 18, wherein the moving visual media depicts an order in which the interpreter viewed the regions preceding designation of the diagnostically relevant landmark.
